(19) United States
(12) Patent Application Publication
(10) Pub. No.: US 2018/0301013 A1
(43) Pub. Date: Oct. 18, 2018

(54) MONITORNING SYSTEMS AND METHODS

(71) Applicant: Shanghai Eagle Safety Equipment Ltd., Shanghai (CN)

(72) Inventors: Lei Wang, Shanghai (CN); Bo Li, Shanghai (CN); Jifei Chen, Shanghai (CN); Ziqiang Huo, Shanghai (CN); Zhiqiang Liu, Shanghai (CN)

(21) Appl. No.: 15/771,121

(22) PCT Filed: Oct. 26, 2015
(86) PCT No.: PCT/CN2015/092821

§ 371 Date: Apr. 26, 2018

(51) Int. Cl. G08B 21/14; G06F 1/16; G01N 33/00

(52) U.S. Cl. CPC G08B 21/14; G01N 33/0075; G06F 1/1632

(57) ABSTRACT

A monitoring system and method include at least one monitor including an internal communication unit, and at least one mobile device that is in communication with the internal communication unit of the monitor. The monitor(s) transmits monitoring data to the mobile device(s) through the internal communication unit. The system and method may also include an external communication unit that is separate and distinct from the monitor(s). The external communication unit receives the monitoring data from the monitor(s) and relays the monitoring data to the mobile device(s).

### FIELD OF THE DISCLOSURE

Embodiments of the present disclosure generally relate to monitoring systems and methods, such as gas or thermal imaging monitoring systems and methods.

### BACKGROUND OF THE DISCLOSURE

Gas sensors or monitors are used to measure concentrations of target gases within particular locations. Personal or portable gas sensors, detectors, or monitors (“personal gas monitors”) are used in various settings to detect hazardous gases. For example, fire and emergency personnel may wear or carry a personal gas monitor in hazardous areas to detect toxic gases, such as carbon monoxide. The personal gas monitor typically includes a gas-detecting medium that is operatively connected to an alarm or display. If the detected gas exceeds an unsafe threshold, an audible alarm may be emitted, and/or a visual alarm may be shown on a display.

One known personal gas monitor includes an internal long range wireless communication system. However, housing such a communication system within a personal gas monitor causes the gas monitor to be large and bulky. Accordingly, the gas monitor may be difficult to wear and/or hold.

### SUMMARY OF THE DISCLOSURE

Certain embodiments of the present disclosure provide a monitoring system that may include at least one monitor including an internal communication unit, and at least one mobile device that is in communication with the internal communication unit. The monitor(s) transmits monitoring data to the mobile device(s) through the internal communication unit. In at least one embodiment, the internal communication unit selectively connects to and disconnects from the mobile device(s), such as through a Universal Serial Bus (USB) interface.

The monitoring system may also include an external communication unit that is separate and distinct from the monitor(s). The external communication unit receives the monitoring data from the monitor(s) and relays the monitoring data to the mobile device(s). Accordingly, the external communication unit may provide a communication bridge between two separate and distinct portable devices, such as the monitor and the mobile device. In at least one embodiment, the external communication unit includes a near field transceiver and a long range transceiver. The external communication unit may wirelessly communicate with the internal communication unit through a near field communication protocol. The external communication unit may wirelessly communicate with the mobile device(s) through a long range communication protocol. For example, the near field communication protocol may be Bluetooth, while the long range communication protocol may be WiFi. The external communication unit may wirelessly communicate with the internal communication unit and the mobile device(s). The external communication unit may electively connect to and disconnect from the mobile device, such as through a USB interface.

In at least one embodiment, the monitor includes a personal gas monitor that includes a gas sensor that detects one or more gases. In at least one other embodiment, the monitor includes a thermal imaging system that includes a thermal infrared red camera.

In at least one embodiment, a single mobile device receives monitoring data from a plurality of monitors. In at least one embodiment, the system may include a central monitoring center in communication with a plurality of mobile devices. Each of the mobile devices may be in communication with a respective group of monitors.

Certain embodiments of the present disclosure provide a monitoring method that may include disposing an internal communication unit within at least one monitor, transmitting monitoring data with the internal communication unit, and receiving the monitoring data with mobile device(s). The method may include selectively connecting and disconnecting the internal communication unit with respect to the mobile device(s). The method may include receiving the monitoring data from the internal communication unit with an external communication unit that is separate and distinct from the at least one monitor, and relaying the monitoring data from the external communication unit to the mobile device(s).

### BRIEF DESCRIPTION OF THE DRAWINGS

FIG. 1 illustrates a front view of a personal gas monitor, according to an embodiment of the present disclosure.

FIG. 2 illustrates a schematic diagram of a monitoring system that is used to communicate information between a personal gas monitor and a mobile device, according to an embodiment of the present disclosure.

FIG. 3 illustrates a schematic diagram of an external communication unit, according to an embodiment of the present disclosure.

FIG. 4 illustrates a schematic diagram of a personal gas monitor, according to an embodiment of the present disclosure.

FIG. 5 illustrates a flow chart of a method of communicating information from a personal gas monitor to a mobile device through an external communication unit, according to an embodiment of the present disclosure.

FIG. 6 illustrates a front view of a monitoring system, according to an embodiment of the present disclosure.

FIG. 7 illustrates a flow chart of a method of communicating information from a personal gas monitor to a mobile device, according to an embodiment of the present disclosure.

FIG. 8 illustrates a front view of a monitoring system, according to an embodiment of the present disclosure.

FIG. 9 illustrates a front view of a monitoring system, according to an embodiment of the present disclosure.

FIG. 10 illustrates a front view of a monitoring system, according to an embodiment of the present disclosure.

### DETAILED DESCRIPTION OF THE DISCLOSURE

The foregoing summary, as well as the following detailed description of certain embodiments, will be better understood when read in conjunction with the appended drawings. As used herein, an element or step recited in the singular and preceded by the word “a” or “an” should be understood as not necessarily excluding the plural of the elements or steps. Further, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Certain embodiments of the present disclosure provide a monitoring system (such as a gas detection system, a thermal imaging system, or the like) that may include a monitor (such as a personal gas monitor or a thermal imaging camera) including a sensor (such as a gas sensor) in communication with an internal communication unit, which may be housed within the monitor. The sensor may be or include a gas sensor that is configured to detect a level of one or more gases. An external communication unit (which is external to the monitor) may be configured to wirelessly communicate with the internal communication unit using a near field communication protocol. The external communication unit may also be configured to wirelessly communicate with a mobile device (such as a smart device, smart phone, or the like) using a long range communication protocol. The external communication unit may be removably secured to an exterior of the monitor. The external communication unit may be within a near field communication range of the internal communication module. The near field communication protocol may be Bluetooth, for example, while the long range communication protocol may be WiFi, for example. The external communication unit may include a near field transceiver and a long range transceiver.

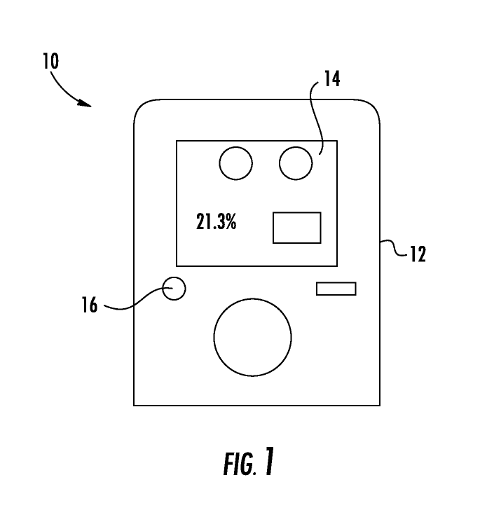

FIG. 1 illustrates a front view of a personal gas monitor 10, according to an embodiment of the present disclosure. The personal gas monitor 10 is an example of a monitor that is used to sense, detect, record, or otherwise monitor one or more attributes of an environment, location, area, or the like. For example, the personal gas monitor 10 is configured to detect a concentration, level, presence, or the like of one or more gases within a location surrounding the personal gas monitor 10.

The personal gas monitor 10 includes a housing 12 that is configured to be worn by an individual, such as on a belt, and/or held by the individual. The housing 12 contains a gas sensor (not shown in FIG. 1), one or more processing circuits (not shown in FIG. 1), and an internal communication unit (not shown in FIG. 1). The housing 12 may include a display 14 configured to show information regarding a detected amount of one or more gases. A gas intake port 16 may be formed through the housing 12 and is in fluid communication with the gas sensor. The personal gas monitor 10 may be various shapes and sizes, other than shown. The personal gas monitor 10 may include a speaker configured to emit audible signals, such as alarms. Alternatively, the personal gas monitor 10 may not include the display 14.

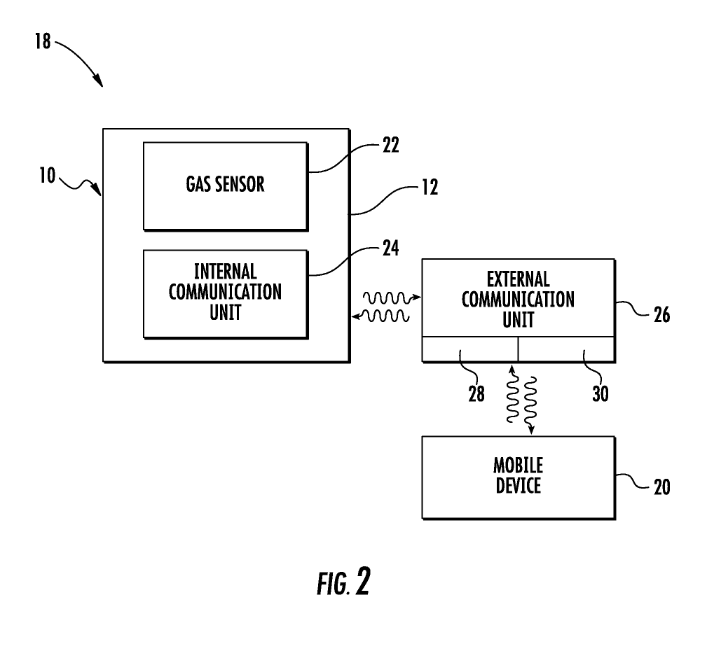

FIG. 2 illustrates a schematic diagram of a monitoring system 18 that is configured to communicate information between the personal gas monitor 10 and a mobile device 20, according to an embodiment of the present disclosure. As shown, the personal gas monitor 10 may include a gas sensor 22, such as an electrochemical gas sensor, that is configured to detect the presence of one or more gases. The gas sensor 22 is in communication with an internal communication unit 24. An external communication unit 26 is outside of the gas monitor 10. For example, the external communication unit 26 may be clipped to the housing 12. Optionally, the external communication unite 26 may be separated from the personal gas monitor 10 within a communication range of the internal communication unit 24. As an example, the gas monitor 10 may be clipped to a belt of an individual, while the external communication unit 26 may be placed in a pants pocket of the individual. The external communication unit 26 is configured to communicate with the mobile device 20 (for example, a handheld smart device and/or smart phone, such as an Apple iPhone), another mobile device, a computer, the cloud, or the like, that is separate and distinct from the personal gas monitor 10.

In operation, the gas sensor 20 of the personal gas monitor 10 detects a level of gas. Information regarding the gas level (for example, monitoring data) is received by the internal communication unit 24, which may then wirelessly transmit the information to the external communication unit 26. The external communication unit 26 may then relay and transmit the information received from the internal communication unit 24 to the mobile device 20. The mobile device 20 may then display information regarding the gas level at the location of the gas monitor 10.

The internal communication unit 24 may be or otherwise include a near field wireless unit, such as a Bluetooth unit. The external communication unit 26 may be or include a near field transceiver 30, such as a Bluetooth transceiver, that receives the information or data from the internal communication unit 24. The external communication unit 26 may also include a long range wireless transceiver 30 that is configured to wirelessly transmit the information or data to the mobile device 20 over a long distance.

As shown, the external communication unit 26 may be separate and distinct from the personal gas monitor 10. The external communication unit 26 may not be contained within the housing 12 of the personal gas monitor 10, thereby ensuring that the personal gas monitor 10 is easy to wear and handle (e.g., the personal gas monitor 10 is not large and bulky).

The external communication unit 26 may alternatively be mechanically and electrically secured to the personal gas monitor 10. In this embodiment, the external communication unit 26 may be powered by a source of power, such as a battery, contained within the personal gas monitor 10. If, however, the external communication unit 26 is separated from the personal gas monitor 10, the external communication unit 26 may include its own source of power, such as a separate and distinct battery.

Referring to FIGS. 1 and 2, the external communication unit 26 provides a wireless bridge between the personal gas monitor 10 and the mobile device 20. The external communication unit 26 may include a separate and distinct housing that is permanently or removably secured to the housing 12. Optionally, the external communication unit 26 may be separated from the personal gas monitor 10 within the near field communication range of the internal communication unit 24. For example, the personal gas monitor 10 may be secured to a belt of an individual, while the external communication unit 26 may be placed in a pants pocket of the individual.

The external communication unit 26 may include the near field transceiver 28, which is configured to communicate with the internal communication unit 24, such as by using the Bluetooth protocol. The external communication unit 26 may also include the long range wireless transceiver 30, which may be used to transmit information or data to the mobile device 20, or another communication bridge within a network.

The near field wireless protocol may be or include Bluetooth, IrDA, NFC, RFID, and/or the like. The long range wireless protocol may be or include Zigbee, WiFi, cellular, and/or the like.

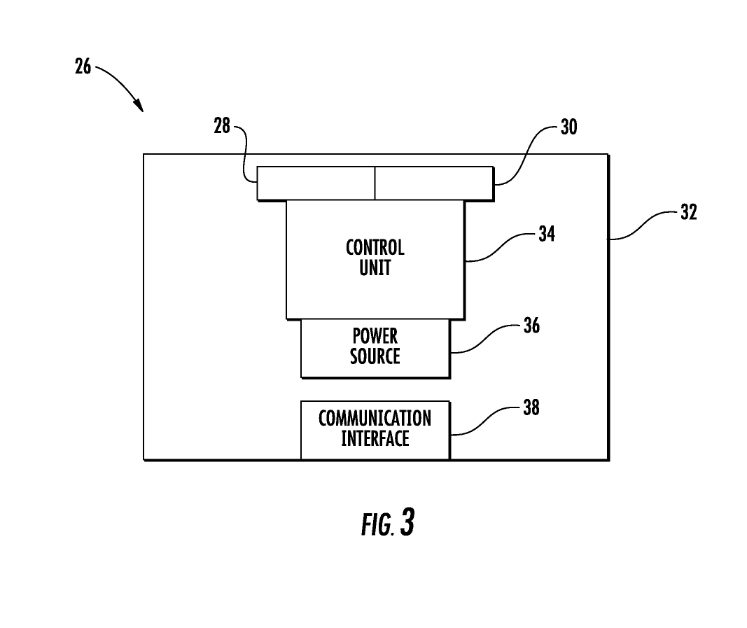

FIG. 3 illustrates a schematic diagram of the external communication unit 26, according to an embodiment of the present disclosure. The external communication unit 26 may include a housing 32 that contains a control unit 34 that is operatively coupled to the near field transceiver 28, the long range wireless transceiver 30, a power source 36, and a communication interface 38. The control unit 34 controls operation of the external communication unit 26. The power source 36 may include one or more batteries (such as rechargeable batteries) that provide power to the external communication unit 26. The communication interface 38 may be a separate and distinct interface that is configured to allow the external communication unit 26 to be directly connected to another device. For example, the communication interface 38 may be a universal serial bus (USB) port, plug, link, cable, and/or the like.

While not shown, the external communication unit 26 may include additional components. For example, the external communication unit 26 may include additional communication interfaces, transceivers, or the like that are configured to communicate over one or more additional communication protocols.

As described with respect to FIG. 2, the external communication unit 26 is configured to operate as a communication bridge between the personal gas monitor 10 and the mobile device 20. For example, the external communication unit 26 is configured to receive and transmit data from the personal gas monitor 10 to the mobile device 20, such as a handheld smart device or smart phone. In at least one other embodiment, the external communication unit 26 may be configured to provide a communication bridge between the mobile device 20 and another monitoring system, such as a thermal imaging system, which may include a heat sensing camera, infrared goggles, and/or the like.

As used herein, the term “controller,” “control unit,” “central processing unit,” “CPU,” “computer,” or the like may include any processor-based or microprocessor-based system including systems using microcontrollers, reduced instruction set computers (RISC), application specific integrated circuits (ASICs), logic circuits, and any other circuit or processor capable of executing the functions described herein. Such are exemplary only, and are thus not intended to limit in any way the definition and/or meaning of such terms.

The control unit 34, for example, is configured to execute a set of instructions that are stored in one or more storage elements (such as one or more memories), in order to process data. For example, the control unit 34 may include or be coupled to one or more memories. The storage elements may also store data or other information as desired or needed. The storage element may be in the form of an information source or a physical memory element within a processing machine.

The set of instructions may include various commands that instruct the control unit 34 as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software may also include modular programming in the form of object-oriented programming. The processing of input data by the processing machine may be in response to user commands, or in response to results of previous processing, or in response to a request made by another processing machine.

The diagrams of embodiments herein may illustrate one or more control or processing units. It is to be understood that the processing or control units may represent circuit modules that may be implemented as hardware with associated instructions (e.g., software stored on a tangible and non-transitory computer readable storage medium, such as a computer hard drive, ROM, RAM, or the like) that perform the operations described herein. The hardware may include state machine circuitry hardwired to perform the functions described herein. Optionally, the hardware may include electronic circuits that include and/or are connected to one or more logic-based devices, such as microprocessors, processors, controllers, or the like. Optionally, the control units may represent processing circuitry such as one or more of a field programmable gate array (FPGA), an application specific integrated circuit (ASIC), microprocessor(s), a quantum computing device, and/or the like. The circuits in various embodiments may be configured to execute one or more algorithms to perform functions described herein. The one or more algorithms may include aspects of embodiments disclosed herein, whether or not expressly identified in a flowchart or a method.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

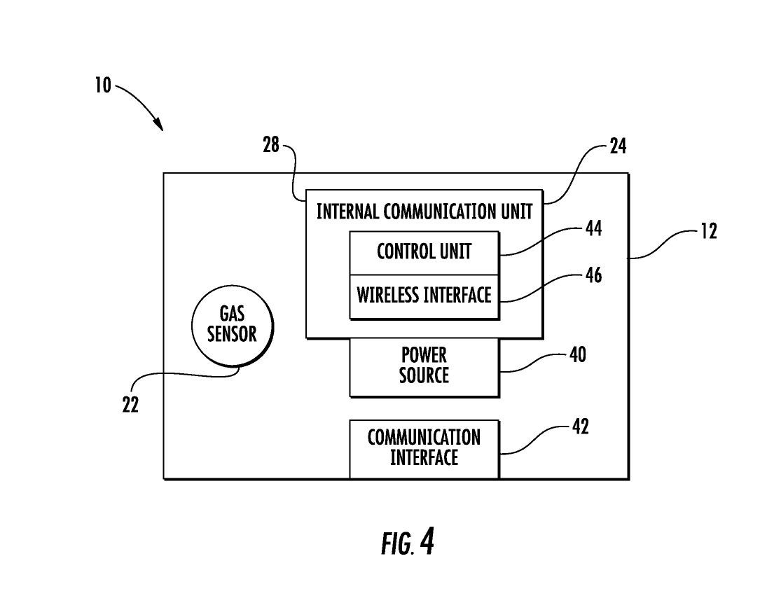

FIG. 4 illustrates a schematic diagram of the personal gas monitor 10, according to an embodiment of the present disclosure. The personal gas monitor 10 includes the housing 12 that contains the gas sensor 22 that is coupled to the internal communication unit 24, a power source 40, and a communication interface 42, such as a universal serial bus (USB) port, plug, link, cable, and/or the like. The power source 40 may be or include or more batteries (such as rechargeable batteries) that provide power for operation of the personal gas monitor 10.

The internal communication unit 24 may include a control unit 44 operatively coupled to the gas sensor 22 and a wireless interface 46, such as a WiFi, Zigbee, Bluetooth, or the like interface. While not shown, the personal gas monitor 10 may include additional components. For example, the internal communication unit 24 may include additional communication interfaces, transceivers, or the like that are configured to communicate over one or more additional communication protocols.

As described with respect to FIG. 2, the internal communication unit 24 is configured to transmit monitoring data (such as gas level information) detected by the gas sensor 22 to the mobile device 20 through the external communication unit 26. For example, the external communication unit 26 is configured to receive and transmit data from the personal gas monitor 10 to the mobile device 20, such as a handheld smart device or smart phone. In at least one other embodiment, the personal gas monitor 10 may be a different type of monitoring system, such as a thermal imaging system.

As noted, the term “controller,” “control unit,” “central processing unit,” “CPU,” “computer,” or the like may include any processor-based or microprocessor-based system including systems using microcontrollers, reduced instruction set computers (RISC), application specific integrated circuits (ASICs), logic circuits, and any other circuit or processor capable of executing the functions described herein. Such are exemplary only, and are thus not intended to limit in any way the definition and/or meaning of such terms.

The control unit 44, for example, is configured to execute a set of instructions that are stored in one or more storage elements (such as one or more memories), in order to process data. For example, the control unit 44 may include or be coupled to one or more memories. The storage elements may also store data or other information as desired or needed. The storage element may be in the form of an information source or a physical memory element within a processing machine.

The set of instructions may include various commands that instruct the control unit 44 as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software may also include modular programming in the form of object-oriented programming. The processing of input data by the processing machine may be in response to user commands, or in response to results of previous processing, or in response to a request made by another processing machine.

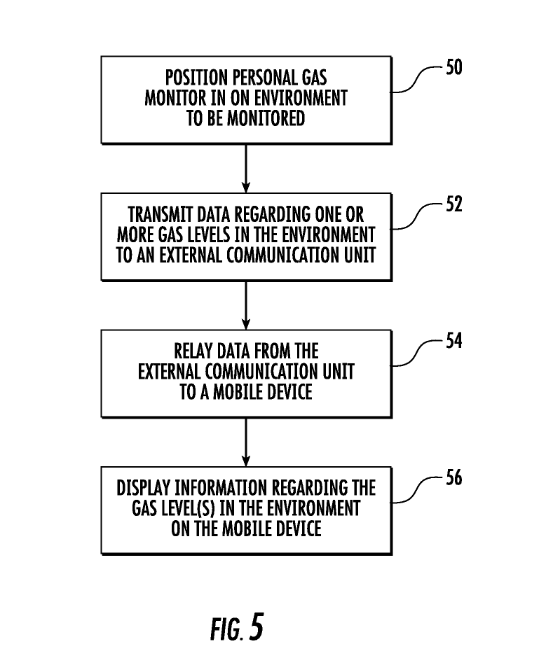

FIG. 5 illustrates a flow chart of a method of communicating information from a personal gas monitor to a mobile device through an external communication unit, according to an embodiment of the present disclosure. The method begins at 50, in which a personal gas monitor is positioned in an environment to be monitored. For example, the personal gas monitor may be worn by an individual who is within the environment.

At 52, data regarding one or more gas levels in the environment are transmitted from the personal gas monitor to an external communication unit. For example, an internal communication unit of the personal gas monitor may wirelessly transmit the data to the external communication unit, which may receive the data through a near and/or far field transceiver.

At 54, the data received at the external communication is relayed from the external communication unit to a mobile device. For example, one or more transceivers of the external communication unit may pass the data from the personal gas monitor to the mobile device,

At 56, information regarding the gas level(s) in the environment may be displayed on the mobile device. For example, the mobile device may be a smart device or smart phone that includes one or more transceivers or communication interfaces that receive the data from the external communication unit. The mobile device may include a display, such as a touchscreen display, that shows the data thereon, such as by showing the gas level(s) detected by the personal gas monitor in the environment being monitored. The mobile device may be in the environment or at a different location.

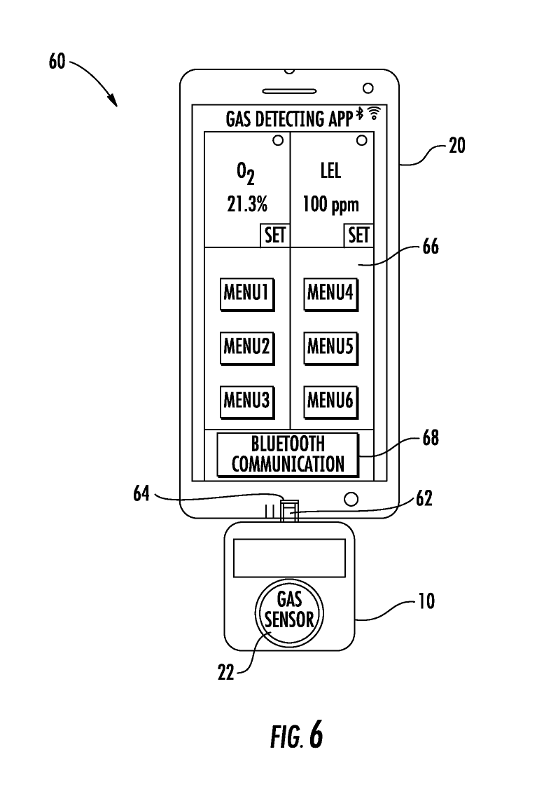

FIG. 6 illustrates a front view of a monitoring system 60, according to an embodiment of the present disclosure. The monitoring system 60 is similar to the monitoring system 18, except that an external communication unit is not used. Instead, the personal gas monitor 10 may be directly connected to the mobile device 20, such as through a USB interface. For example, the personal gas monitor 10 may include a USB plug 62 that is removably connected to a USB port 64 of the mobile device 20.

The mobile device 20 may include a display 66 that is configured to show information regarding gas level(s) detected by the gas sensor 22 of the personal gas monitor 10. For example, the mobile device 20 may include software, such as an application, that is configured to display various characteristics of one or more gases detected by the gas sensor 22.

The personal gas monitor 10 may be selectively connected to (such as by being plugged into) and disconnected from (such as being unplugged from) the mobile device 20. The personal gas monitor 10 may be connected to the mobile device 20 when gas monitoring is desired, and removed from the mobile device 20 after a monitoring period. In this manner, the monitoring system 60 provides an adaptable and efficient system that does not utilize a specialized, bulky housing. Instead, the personal gas monitor 10 is simply selectively connected and disconnected from an existing mobile device 20.

Additionally, the mobile device 20 may include a Bluetooth interface 68 that allows for wireless communication between the personal gas monitor 10 and the mobile device 20. As such, the mobile device 20 need not be directly connected to the mobile device 20 in order to communicate therewith.

Referring to FIGS. 2 and 6, the monitoring system 60 may optionally also include the external communication unit 26. For example, the personal gas monitor 10 may not be within a communication range of the mobile device 20. The external communication unit 26 may provide a communication bridge between the personal gas monitor 10 and the mobile device 20, as described above. In at least one embodiment, the internal communication unit 24 of the personal gas monitor 10 may transmit data to the external communication unit 26 through a first communication protocol, such as Bluetooth. The external communication device 26 may then transmit the data to the mobile device 20 through a direct connection (such as a USB interface), and/or wirelessly through a second communication protocol, such as Wifi, Zigbee, or the like.

If Zigbee, for example, is used as a long range communication protocol, but a mobile device is not configured to communicate via Zigbee, the external communication unit 26 may wirelessly communicate with the internal communication unit 24 through a near field communication protocol. The external communication unit 26 may also wirelessly communicate with a second external communication unit through a far field communication protocol. The second external communication unit may then relay the data to a mobile device through a near field communication protocol

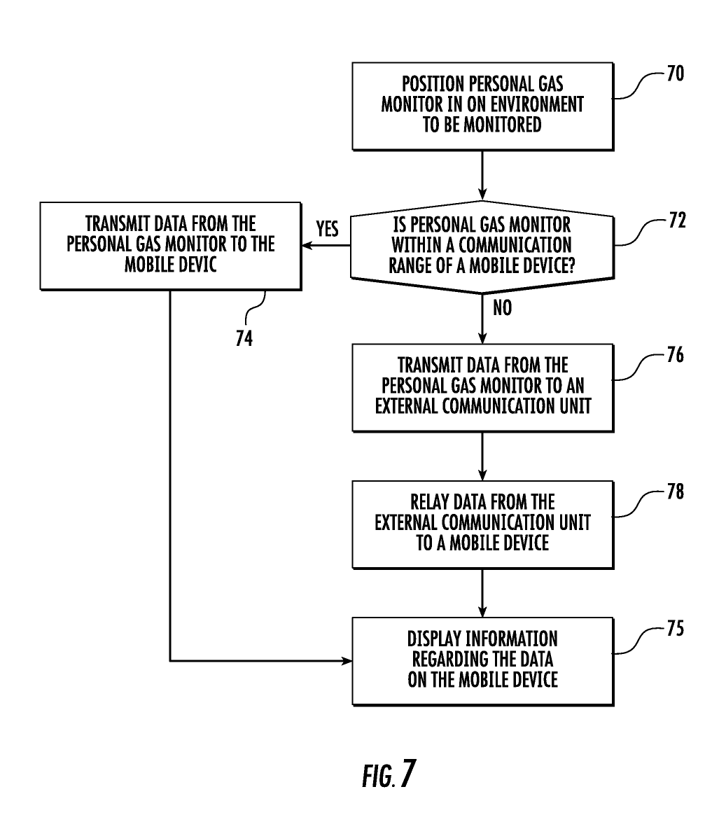

FIG. 7 illustrates a flow chart of a method of communicating information from a personal gas monitor to a mobile device, according to an embodiment of the present disclosure. The method begins at 70, in which a personal gas monitor is positioned within an environment to be monitored. At 72, it is determined whether the personal gas monitor is within a communication range of a mobile device. If so, the method proceeds from 72 to 74, in which data is transmitted from the personal gas monitor to the mobile device. For example, the personal gas monitor may be directly connected to the mobile device through a USB interface and/or within a Bluetooth communication range. Information regarding the data is then displayed on the mobile device at 75.

If, however, the personal gas monitor is not within the communication range of the mobile device, the method proceeds from 72 to 76, in which data is transmitted from the personal gas monitor to the external communication unit, which acts as a wireless communication bridge between the personal gas monitor and the mobile device. Then, at 78, data is relayed from the external communication unit to the mobile device 78. The method then proceeds from 78 to 75.

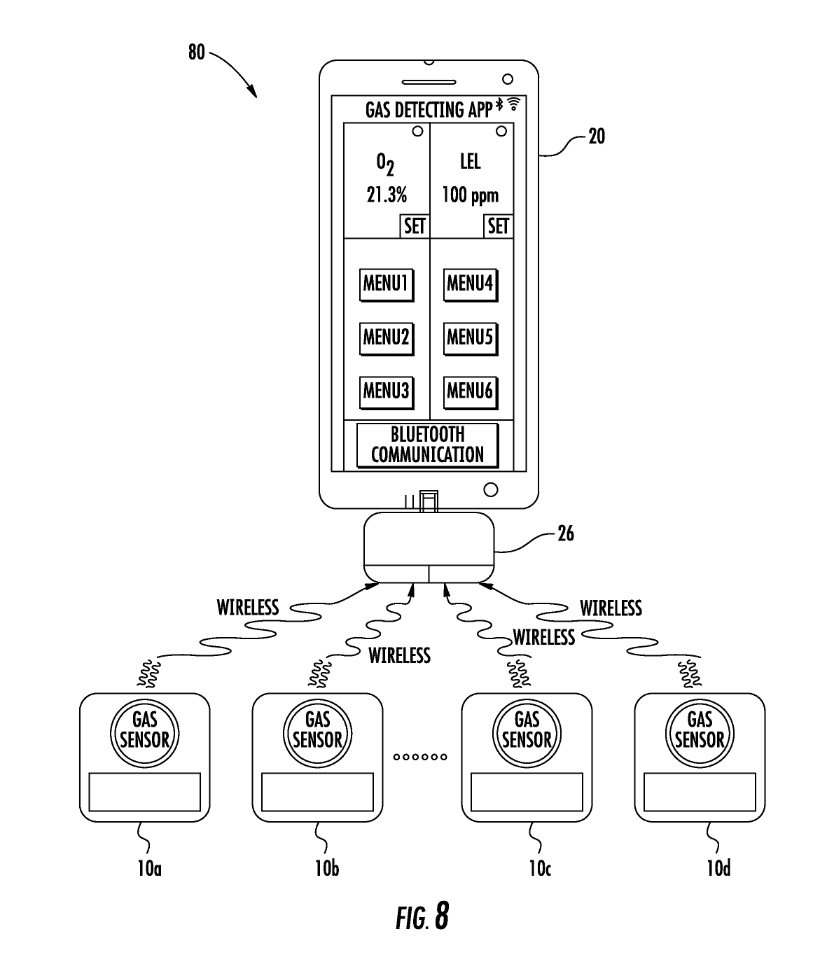

FIG. 8 illustrates a front view of a monitoring system 80, according to an embodiment of the present disclosure. The monitoring system 80 may include the mobile device 20 and the external communication unit 26 in communication with the mobile device 20. For example, the external communication unit 26 may be directly connected to the mobile device 20 through a USB interface.

The external communication unit 26 is in communication with a plurality of personal gas monitors 10a-10n (more or less personal gas monitors than shown may be used). Each of the personal gas monitors 10a-10n wirelessly communicates with the external communication unit 26, as described above. Data received from each of the personal gas monitors 10a-10n may be shown on the mobile device 20. In this manner, the monitoring system 80 may be configured to monitor multiple locations through multiple personal gas monitors 10a-10n. In at least one other embodiment, different gas monitors 10a-10n may be used to monitor different gases.

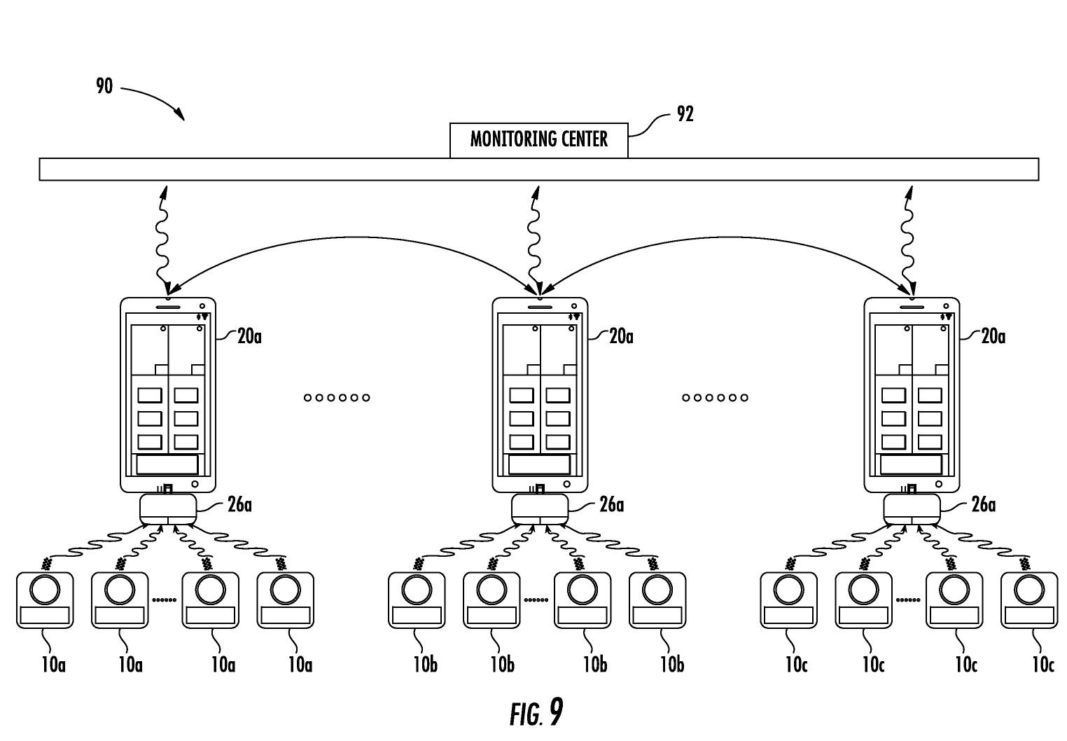

FIG. 9 illustrates a front view of a monitoring system 90, according to an embodiment of the present disclosure. The monitoring system 90 may include a central monitoring center 92 that is in communication with a plurality of remote devices 20a-20n. Each of the remote devices 20a-20n may be coupled to a respective external communication unit 26a-26n, each of which wirelessly communicates with a different set of personal gas monitors 10a, 10b, and 10n. More or less remote devices than shown may be used. Further, more or less personal gas monitors than shown may be used.

The central monitoring center 92 may be, for example, the Cloud, a central server, one or more local servers, and/or the like. The monitoring system 90 provides a monitoring network that is configured to monitor a large number of personal gas monitors, which may communicate with different mobile devices.

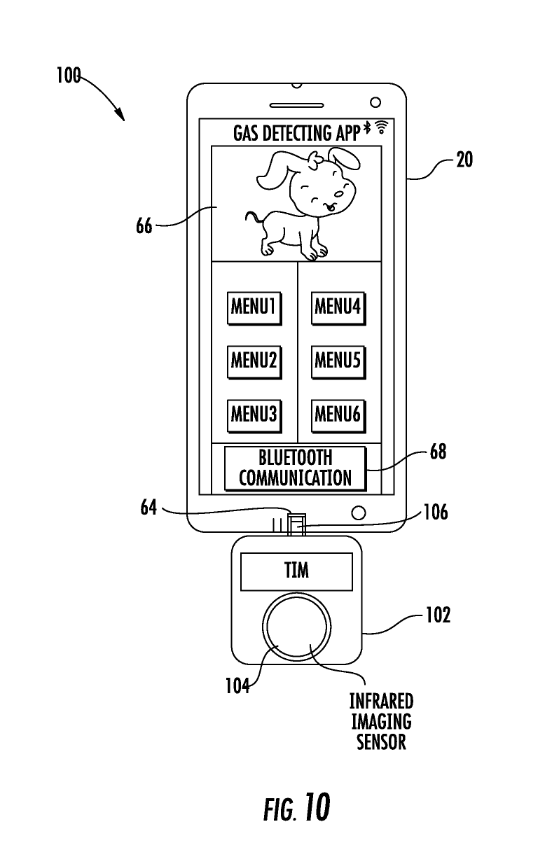

FIG. 10 illustrates a front view of a monitoring system 100, according to an embodiment of the present disclosure. The monitoring system 100 is similar to those described above, except, instead of a personal gas monitor, the monitoring system 100 includes a monitor in the form of a thermal imaging monitor 102, which may include an infrared imaging sensor 104.

As shown, the monitoring system 100 may not include an external communication unit. Instead, the thermal imaging monitor 102 may be directly connected to a mobile device 20, such as through a USB interface. For example, the thermal imaging monitor 102 may include a USB plug 106 that is removably connected to a USB port 64 of the mobile device 20.

The mobile device 20 may include a display 66 that is configured to show thermal images detected by the thermal imaging monitor 102. For example, the mobile device 20 may include software, such as an application, that is configured to display thermal images on the display 66.

The thermal imaging monitor 102 may be selectively connected to and disconnected from the mobile device 20. The thermal imaging monitor 102 may be connected to the mobile device 20 when thermal imaging is desired, and removed from the mobile device 20 after a monitoring period. In this manner, the monitoring system 100 provides an adaptable and efficient system that does not utilize a specialized, bulky housing. Instead, the thermal imaging monitor 102 is simply selectively connected and disconnected from an existing mobile device 20.

Additionally, the mobile device 20 may include a Bluetooth interface 68 that allows for wireless communication between the thermal imaging monitor 102 and the mobile device 20. As such, the mobile device 20 need not be directly connected to the mobile device 20 in order to communicate therewith.

Referring to FIGS. 2 and 10, the monitoring system 100 may optionally also include the external communication unit 26. For example, the thermal imaging monitor 102 may not be within a communication range of the mobile device 20. The external communication unit 26 may provide a communication bridge between the thermal imaging monitor 102 and the mobile device 20, as described above. In at least one embodiment, the thermal imaging monitor 102 may include an internal communication unit 24 and may transmit data to the external communication unit 26 through a first communication protocol, such as Bluetooth. The external communication device 26 may then transmit the data to the mobile device 20 through a direct connection (such as a USB interface), and/or wirelessly through a second communication protocol, such as WiFi, Zigbee, or the like.

Referring to FIGS. 1-10, embodiments of the present disclosure provide monitoring systems that include one or more monitors (such as a personal gas monitor, a thermal imaging monitor, and/or the like) that are configured to efficiently and adaptively communicate with a mobile device. The monitors may not include internal long range wireless communication systems. As such, the monitors are easy to hold, carry, and wear. In at least one embodiment, a monitor may be selectively connected to and disconnected from a mobile device, which may include an application that is configured to display data transmitted from the monitor.

While various spatial and directional terms, such as top, bottom, lower, mid, lateral, horizontal, vertical, front and the like may be used to describe embodiments of the present disclosure, it is understood that such terms are merely used with respect to the orientations shown in the drawings. The orientations may be inverted, rotated, or otherwise changed, such that an upper portion is a lower portion, and vice versa, horizontal becomes vertical, and the like.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described embodiments and/or aspects thereof) may be used in combination with each other. In addition, many modifications may be made to adapt a particular situation or material to the teachings of the various embodiments of the disclosure without departing from their scope. While the dimensions and types of materials described herein are intended to define the parameters of the various embodiments of the disclosure, the embodiments are by no means limiting and are exemplary embodiments. Many other embodiments will be apparent to those of skill in the art upon reviewing the above description. The scope of the various embodiments of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various embodiments of the disclosure, including the best mode, and also to enable persons skilled in the art to practice the various embodiments of the disclosure, including making and using any devices or systems and performing any incorporated methods. The patentable scope of the various embodiments of the disclosure is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

1. A monitoring system comprising:
at least one monitor including an internal communication unit; and
at least one mobile device that is in communication with the internal communication unit of the at least one monitor, wherein the at least one monitor transmits monitoring data to the at least one mobile device through the internal communication unit.

2. The monitoring system of claim 1, wherein the internal communication unit selectively connects to and disconnects from the at least one mobile device.

3. The monitoring system of claim 1, further comprising an external communication unit that is separate and distinct from the at least one monitor, wherein the external communication unit receives the monitoring data from the at least one monitor and relays the monitoring data to the at least one mobile device.

4. The monitoring system of claim 3, wherein the external communication unit includes a near field transceiver and a long range transceiver.

5. The monitoring system of claim 3, wherein the external communication unit wirelessly communicates with the internal communication unit through a near field communication protocol, and wherein the external communication unit wirelessly communicates with the at least one mobile device through a long range communication protocol.

6. The monitoring system of claim 5, wherein the near field communication protocol is Bluetooth, and wherein the long range communication protocol is WiFi or Zigbee.

7. The monitoring system of claim 3, wherein the external communication unit wirelessly communicates with the internal communication unit and the at least one mobile device.

8. The monitoring system of claim 3, wherein the external communication unit selectively connects to and disconnects from the at least one mobile device.

9. The monitoring system of claim 1, wherein the at least one monitor comprises at least one personal gas monitor that includes a gas sensor that detects one or more gases.

10. The monitoring system of claim 1, wherein the at least one monitor comprises at least one thermal imaging system that includes a thermal infrared red camera.

11. The monitoring system of claim 1, wherein the at least one monitor comprises a plurality of monitors, and wherein the at least one mobile device comprises a single mobile device.

12. The monitoring system of claim 1, further comprising a central monitoring center, wherein the at least one monitor comprises a plurality of groups of monitors, wherein the at least one mobile device comprises a plurality of mobile devices in communication with the central monitoring center, and wherein each of the plurality of mobile devices is in communication with a respective one of the plurality of groups of monitors.

13. A monitoring method comprising:
disposing an internal communication unit within at least one monitor;
transmitting monitoring data with the internal communication unit; and
receiving the monitoring data with at least one mobile device.

14. The monitoring method of claim 13, wherein selectively connecting and disconnecting the internal communication unit with respect to the at least one mobile device.

15. The monitoring system of claim 1, further comprising:
receiving the monitoring data from the internal communication unit with an external communication unit that is separate and distinct from the at least one monitor; and
relaying the monitoring data from the external communication unit to the at least one mobile device.

16. The monitoring method of claim 15, wherein the external communication unit includes a near field transceiver and a long range transceiver.

17. The monitoring method of claim 15, wherein the external communication unit wirelessly communicates with the internal communication unit through a near field communication protocol, and wherein the external communication unit wirelessly communicates with the at least one mobile device through a long range communication protocol.

18. The monitoring method of claim 17, wherein the near field communication protocol is Bluetooth, and wherein the long range communication protocol is WiFi or Zigbee.

19. The monitoring method of claim 15, wherein the external communication unit is configured to wirelessly communicate with the internal communication unit and the at least one mobile device.

20. The monitoring method of claim 15, further comprising selectively connecting and disconnecting the external communication unit with respect to the at least one mobile device.

21-24. (canceled)
